(19) United States
(12) Patent Application Publication
(10) Pub. No.: US 2005/0113282 A1
(43) Pub. Date: May 26, 2005

(54) Melamine-formaldehyde microcapsule slurries for fabric article freshening

(76) Inventors: Prabodh P. Parekh, Marlboro, NJ (US); Guillermo H. Fernandez, Lebanon, NJ (US); Kristine K. Colt, Jackson, NJ (US)

Correspondence Address: INTERNATIONAL FLAVORS & FRAGRANCES INC., 521 WEST 57TH ST, NEW YORK, NY, 10019, US

(21) Appl. No.: 10/718,239

(22) Filed: Nov. 20, 2003

(51) Int. Cl. A61K 7/46

(52) U.S. Cl. 512/1

(57) ABSTRACT

Described is a method for freshening fabric articles by means of spraying the articles with an aqueous slurry of microcapsules having rupturable melamine-formaldehyde polymeric walls, containing substantive and efficacious functional substances, e.g., malodour counteractants and/or fragrances. The slurry may optionally contain non-confined functional substances, e.g. malodour counteractants and/or fragrances. The method is effective for the deposition of effectively-rupturable malodour suppressant and/or fragrance emitting microcapsules onto fabrics wherein the resulting emitted fragrance activity and/or malodour counteractant activity is long-lasting and where the resulting substantive aroma is aesthetically pleasing over the long period of time for which it is effective. Also described are efficacious and substantive malodour counteractant compositions useful for the aforementioned process containing a malodour composition composed of zinc ricinoleate and at least one of: 1-cyclohexylethan-1-yl butyrate; 1-cyclohexylethan-1-yl acetate; 1-cyclohexylethan-1-ol; 1-(4′-methylethyl)cyclohexylethan-1-yl propionate; and/or 2′-hydroxy-1′-ethyl(2-phenoxy)acetate. In addition, described are efficacious microcapsule slurries useful for the aforementioned process containing microcapsules having melamine-formaldehyde polymeric capsule walls with the microcapsules being in contact with one or more polymeric silicone phospholipids.

### FIELD OF THE INVENTION

Freshening fabric articles by means of spraying the articles with aqueous slurry of microcapsules having rupturable melamine-formaldehyde polymeric walls, containing substantive and efficacious malodour counteractants and/or fragrances.

### BACKGROUND OF THE INVENTION

The need for application of various functional products, e.g., malodour counteractants to wearable fabrics with at least a modicum of permanency has been well-recognized throughout the history of modern mankind. Thus, fabrics containing imbedded microcapsules composed of melamine-formaldehyde polymeric shells containing fragrances are disclosed in the prior art, for example, U.S. Pat. No. 4,917,920. In addition, processes for spraying fabric wrinkle control agents together with materials which remove or reduce undesirable odor from malodourous fabric are known in the prior art, for example, U.S. Pat. Nos. 6,001,343 and 6,146,621. In addition, methods for spraying functional product-containing microcapsules, for example, fragrance-containing microcapsules, onto fabrics are disclosed in the prior art, U.S. Pat. No. 6,071,569. Furthermore, methods for spraying microcapsules such as those composed of melamine-formaldehyde polymeric shells containing fragrances, including malodour-counteracting fragrances onto substrates such as floors are also suggested in the prior art, for example, PCT Published Patent WO 02/085420 A1. However, nothing set forth in the prior art either explicitly or implicitly describes a technique for the permanent deposition of effectively-rupturable malodour suppressant and/or fragrance emitting microcapsules onto fabrics wherein the resulting emitted fragrance activity and/or malodour counteractant activity is long-lasting and where the resulting substantive aroma is aesthetically pleasing over the long period of time for which it is effective.

U.S. patent application Ser. No. 10/706,888 filed on Nov. 13, 2003 discloses the synergistic malodour counteractant composition of zinc ricinoleate and substituted monocyclic organic compounds such as 1-cyclohexylethan-1-yl butyrate but does not disclose the use of such compositions in microencapsulated form or in aqueous slurries.

### SUMMARY OF THE INVENTION

Our invention is directed to a process for freshening fabric articles by means of spraying the articles with an aqueous slurry of microcapsules each of which microcapsule has a rupturable melamine-formaldehyde polymeric wall, containing substantive and efficacious malodour counteractants and/or fragrances and to compositions of matter which include microencapsulated products useful in connection with the carrying out of the aforementioned process.

More specifically, our invention is directed to a method for substantively (a) imparting fragrance to and/or (b) substantially eliminating malodours from and/or (c) covering malodours evolved from and/or (d) preventing malodour formation in at least one fabric article for an extended period of time comprising the steps of:
i. providing one or more exposed surface areas of one or more fabric articles;
ii. providing an enclosure equipped with at least one pressure-activated air atomizer having an externally-located spray nozzle communicating with the interior of said enclosure, said nozzle having from 1 up to a plurality of nozzle exit ports each of which has a nozzle exit port effective diameter, D_{NPi};
iii. preparing a plurality of microcapsules each of which is composed of a rupturable external wall of a melamine-formaldehyde polymer enclosing from about 10 weight % to about 30 weight % of a first functional substance which is one or more of (a) a fragrance composition each of the components of which has a C log₁₀P of between 2.5 and 8, wherein P is the n-octanol/water partition coefficient of said component; (b) a malodour counteracting composition each of the components of which has a C log₁₀OP of between 2.5 and 8, wherein P is the n-octanol/water partition coefficient of said component; and/or (c) a malodour-preventing composition each of the components of which has a C log₁₀P of between 2.5 and 8, wherein P is the n-octanol/water partition coefficient of said component, each of said microcapsules having an average effective diameter \overline{D_{MC}} of from about 5 microns to about 80 microns, each of which microcapsule has an effective diameter of D_{MCi} wherein the smallest of D_{NPi} is substantially greater than the greatest of D_{MCi} wherein

$$\overline{{D}_{MC}} = \frac{1}{N} {ΣD}_{MCi}$$

and N represents the number of microcapsules in the N slurry contained in said enclosure;
iv. providing an aqueous solution comprising (a) water, (b) a compatible solvent which is one or more of ethanol, the mono-C₁ or C₂ ether of a mono-, di-, or tri-1,2-propylene glycol and/or the di-C₁ or C₂ ether of a mono-, di- or tri-1,2-propylene glycol, (c) a compatible silicone polymer, (d) a compatible non-ionic surfactant, (e) a compatible preservative and (f) a compatible suspending agent;
v. admixing said plurality of microcapsules with said aqueous solution at a level of from about 0.1 weight % to about 0.4 weight % of microcapsules based on the weight of aqueous solution, thereby forming a microcapsule slurry wherein said microcapsules are suspended in said slurry and each of said microcapsules has a settling velocity in said slurry, V_{S} equal to about 0;
vi. optionally causing a non-encapsulated second functional substance which is one or more of (a) a fragrance composition each of the components of which has a C log₁₀P of between 1 and 8, wherein P is the n-octanol/water partition coefficient of said component; (b) a malodour counteracting composition each of the components of which has a C log₁₀P of between 1 and 8, wherein P is the n-octanol/water partition coefficient of said component; and/or (c) a malodour-preventing composition each of the components of which has a C log₁₀P of between 1 and 8, wherein P is the n-octanol/water partition coefficient of said component to be in admixture with said slurry by means of admixing said second functional composition (A) with said aqueous solution and/or (B) with said slurry;
vii. placing said microcapsule slurry into said enclosure;
viii. situating said enclosure whereby the nozzle exit ports of the externally-located spray nozzle are each substantially located in a plane substantially parallel to and opposite said one or more exposed surface areas of said one or more fabric articles at a substantially perpendicular mean distance of from about 0 to about 3 meters from said one or more exposed surface areas of said one or more fabric articles;
ix. applying sufficient pressure to said slurry located within said enclosure to enable said slurry to be sprayed through said one or more nozzle exit ports onto said one or more exposed surface areas of said one or more fabric articles whereby said microcapsules are effectively adhered to said one or more exposed surface areas of said one or more fabric articles thereby forming one or more microcapsule-fixed fabric article surface areas; and
   whereby (a) the concentration of the functional substance contained in the slurry is from about 0.03% to about 0.8%, preferably from about 0.05% to about 0.3% immediately prior to the step ix and (b) subsequent to the step ix when said microcapsule-fixed fabric article surface areas are rubbed, said microcapsules rupture, thereby emitting said first functional substance.

The aforementioned first functional substance and/or second functional substance may comprise a mixture of zinc ricinoleate or a solution thereof and a substituted monocyclic organic compound which is in the alternative or in combination one or more of:
1-cyclohexylethan-1-yl butyrate;
1-cyclohexylethan-1-yl acetate;
1-cyclohexylethan-1-ol;
1-(4′-methylethyl)cyclohexylethan-1-yl propionate; and
   2′-hydroxy-1′-ethyl(2-phenoxy)acetate
      each of which compound is marketed under the trademark VEILEX by International Flavors & Fragrances Inc., New York, N.Y., U.S.A.

Capsules having walls composed of a melamine-formaldehyde polymer containing such zinc ricinoleate-containing mixtures are novel compositions of matter.

Useful in the practice of our invention are microcapsules each of which is composed of a melamine-formaldehyde polymeric shell where there is enclosed within the shell a functional ingredient which is either (a) a fragrance composition each of the components of which has a C log₁₀P of between 2.5 and 8, wherein P is the n-octanol/water partition coeffiicent of said component, and/or (b) a malodour counteracting composition each of the components of which has a C log₁₀P of between 2.5 and 8, with the microcapsule being in intimate contact with one or more polymeric silicone phospholipids which is(are) included in the microcapsule slurry at a rate of 0.05% to 0.8%, preferably 0.1-0.3% and most preferably, 0.2%.

Preferably, such polymeric silicone phospholipid(s) is(are) prepared by the phosphation reaction of a terminal dimethicone copolyol with a phosphating agent followed by neutralization of the phosphate with base followed by a condensation reaction with an epihalohydrin followed by conducting a n-alkylation reaction with an amine.

In addition, all or a portion of the microcapsules useful in the practice of our invention may be coated with coatings as described in Applications for U.S. Letters patent Ser. Nos. 10/268,566 and/or 10/268,526 each having been filed on October 10, 2002.

### DETAILED DESCRIPTION OF THE INVENTION

In carrying out the process of our invention, the enclosure which is equipped with a pressure-activated air atomizer is a spray dispenser which, most preferably is a manually or non-manually activated pump-spray dispenser for containing the slurry suspension to be applied to fabric articles in accordance with the process of our invention. A number of trigger sprayers or finger pump sprayers are suitable for carrying out the process of our invention, for example, the Indesco T-8500 sprayer available from Continental Sprayers, Inc. of St. Peters, Missouri, U.S.A. Other useful sprayers are described in U.S. Pat. Nos. 4,082,223 and 4,819,835. Other useful sprayers for carrying out the process of our invention are disclosed in U.S. Pat. No. 6,592,813.

Methods for preparing the functional product-encapsulates having melamine formaldehyde polymeric shells useful in the practice of our invention are disclosed in the prior art according to U.S. Pat. Nos. 3,516,846; 4,681,806; 6,024,943; 6,194,375; 6,413,548; Published U.S. Patent Application 2003/0125222A1 and Published U.S. Patent Application 2003/0199412A1. The particle size range of the encapsulates useful in carrying out the process of our invention is in the range of 5-80 microns; preferably 5-40 microns; and most preferably 5-10 microns.

The functional product, e.g., fragrance and/or malodour counteractant encapsulated in the melamine-formaldehyde polymeric encapsulate used in the practice of our invention has a C log₁₀P of between 2.5 and 8, as stated above.

The values of C log₁₀P of many functional product ingredients, for example, fragrance ingredients contained in personal treatment compositions and/or cosmetic compositions is discussed in U.S. Pat. Nos. 5,968,404 and 6,495,058. Furthermore, values of log₁₀P have been reported; for example, the Pomona92 database, available from Daylight Chemical Information Systems, Inc., Daylight CIS, Irvine, Calif. However, the log₁₀P values are most conveniently calculated by the “CLOGP” program, also available from Daylight CIS. This program also lists experimental log₁₀P values when they are available in the Pomona92 database. The “calculated log₁₀P” (C log₁₀P) is determined by the Hansch and Leo “fragment” approach based on the chemical structure of each functional product ingredient, and takes into account the numbers and types of atoms, the atom connectivity and the chemical bonding. The C log₁₀P values which are the most reliable and widely used estimates for this physicochemical property, are preferably used instead of the experimental log₁₀P values for the selection of functional ingredients, including perfume ingredients which are useful components in the microencapsulate-containing slurries of our invention.

Specific examples of preferred fragrance components useful in melamine-formaldehyde polymeric microencapsulates used in the process of our invention, and the molecular weights and C log₁₀P values of each of said components are set forth in Table I as follows:

TABLE I

| | Clog₁₀P | Molecular |
|---|---|---|
| Fragance Component | value | Weight |
| amyl salicylate | 4.601 | 208.26 |
| benzyl salicylate | 4.383 | 228.25 |
| β-caryophyllene | 6.333 | 204.36 |
| ethyl undecylenate | 4.888 | 212.34 |
| geranyl anthranilate | 4.216 | 273.38 |
| α-irone | 3.820 | 206.33 |
| β-phenyl ethyl benzoate | 4.058 | 226.28 |
| 1-phenyl hexanol-5 | 3.299 | 178.28 |
| α-santalol | 3.800 | 220.36 |
| amyl salicylate | 4.601 | 208.26 |
| β-caryophyllene | 6.333 | 204.36 |
| cedrol | 4.530 | 222.37 |
| cedryl acetate | 5.436 | 264.41 |
| cedryl formate | 5.070 | 238.37 |
| cyclohexyl salicylate | 5.265 | 220.29 |
| γ-dodecalactone | 4.359 | 198.31 |
| β-phenylethyl phenyl acetate | 3.767 | 240.31 |
| 5-acetyl-1,1,2,3,3,6-hexamethyl | 5.977 | 258.41 |
| indane | | |
| cyclopentadecanolide | 6.246 | 240.39 |
| d-limonene | 4.232 | 136.24 |
| amyl cinnamic aldehyde | 4.324 | 202.30 |
| linalyl benzoate | 5.233 | 258.36 |

The aqueous solution provided in step iv of the process of our invention is composed of components which do not detract from the desired effects of carrying out the process of our invention on fabric articles. For example, each components of such aqueous solution are to be substantially free of any substantially-detectable disagreeable aroma and no component is to leave any discernable residue subsequent to the drying of the product applied to the fabric article as a result of the carrying out of the process of our invention. Accordingly, in the process of our invention the aqueous solution provided in step iv, above, preferably consists essentially of (a) from about 80 to about 93 parts by weight of water; (b) from about 4 to about 8 parts by weight of ethanol or a methyl and/or ethyl ether of propylene glycol, di-propylene glycol and/or tripropylene glycol, with use of materials such as methyl cellosolve is contraindicated in view of the aroma thereof; (c) from about 2 to about 3 parts by weight of a compatible non-ionic surfactant; (d) from about 0.05 to about 0.5 parts by weight of a compatible preservative; (e) from about 0.1 to about 2 parts by weight of a compatible silicone polymer and (f) from about 0.05 to about 0.1 parts by weight of compatible suspending agent.

Preferred silicone polymers are emulsified silicones having the formula:
(CH₃)₃SiO[(CH₃)₂SiO]ₘSi(CH₃)₂
wherein m is in the range of from 1 to 8. Other useful silicone polymers are polydimethyl siloxanes as disclosed in U.S. Pat. No. 6,001,343 at Column 29, lines 1-25.

In the aqueous solution used in the process of our invention the non-ionic surfactant is preferred to be a mixture of the hydroxy-octaethoxy ethers of n-nonanol and n-undecanol (TOMADOL 91-8, trademark of Tomah Products, Inc.)., the compatible preservative is preferably hexahydro-1,3,5-tris(2-hydroxyethyl)-s-triazine (SURCIDE P available from Surety Laboratories) and the compatible suspending agent is preferably one or more of attapulgite clay, xanthan gum, hydroxypropyl cellulose having a molecular weight of from about 50,000 to about 800,000, colloidal silica and/or ethyl cellulose having a particle size of from about 0.004 microns to about 0.130 microns, a surface area of from about 100 m² per gram to about 500 m² per gram and a density of from about 1.0 to about 4.0 pounds per ft³.

For purposes of substantivity enhancement of the delivery to the fabric article of the-microencapsulated functional product in accordance with the process of our invention, the slurry containing the melamine-formaldehyde polymeric shells encapsulating the aforementioned fragrance and/or malodour suppressing or counteracting or preventing components may be in admixture with a silicone phospholipid polymer additive as exemplified in U.S. Pat. No. 5,237,035, examples 56-85 at column 12 thereof. Such silicone phospholipid polymer additive(s) are included in the microcapsule slurry useful in the practice of our invention at a rate of 0.05% to 0.8%, preferably 0.1-0.3% and most preferably, 0.2%. Such slurries of containing the silicone phospholipid polymers are novel compositions of matter.

In addition, synergistic combinations of malodour counteractants may be included in the microencapsulated compositions useful in the practice of our invention, for example, the compositions comprising:
(i) from about 10 to about 90 parts by weight of at least one substituted monocyclic organic compound-containing material which is:
   (a) 1-cyclohexylethan-1-yl butyrate having the structure: 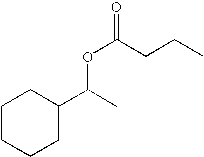
   (b) 1-cyclohexylethan-1-yl acetate having the structure: 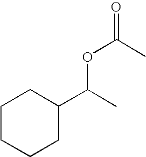
   (c) 1-cyclohexylethan-1-ol having the structure: 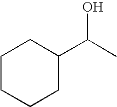
   (d) 1-(4′-methylethyl)cyclohexylethan-1-yl propionate having the structure: 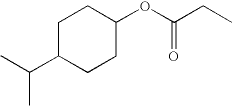
      and
   (e) 2′-hydroxy-1′-ethyl(2-phenoxy)acetate having the structure: 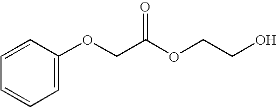
      and (ii) from about 90 to about 10 parts by weight of a zinc ricinoleate-containing composition which is zinc ricinoleate and/or solutions of zinc ricinoleate containing greater than about 30% by weight of zinc ricinoleate. Preferably, the aforementioned zinc ricinoleate-containing compositions are mixtures of about 50% by weight of zinc ricinoleate and about 50% by weight of at least one 1-hydroxy-2-ethoxyethyl ether of a C₁₂-C₁₄ fatty alcohol.

More specifically, a preferred composition useful in combination with the zinc ricinoleate component is a mixture of:
(A) 1-cyclohexylethan-1-yl butyrate;
(B) 1-cyclohexylethan-1-yl acetate; and
(C) 1-(4′-methylethyl)cyclohexylethan-1-yl propionate.

More preferably, the weight ratio of components of the immediately-aforementioned zinc riconoleate-containing mixture is one where the zinc ricinoleate-containing composition:

1-cyclohexylethan-1-yl butyrate: 1-cyclohexylethan-1-yl acetate: 1-(4′-methylethyl)-cyclohexylethan-1-yl propionate is about 2:1:1:1.

Another preferred composition useful in combination with the zinc ricinoleate component or solution is a mixture of:
(A) 1-cyclohexylethan-1-yl acetate; and
(B) 1-(4′-methylethyl)cyclohexylethan-1-yl propionate.

More preferably, the weight ratio of components of the immediately-aforementioned zinc riconoleate mixture is one where the zinc ricinoleate-containing composition: 1-cyclohexylethan-1-yl acetate: 1-(4′-methylethyl)cyclohexylethan-1-yl propionate is about 3:1:1.

Optionally, the slurry used in practicing the process of our invention may also contain non-confined (or “non-encapsulated”) functional product, e.g., fragrance, each of the components of which has a C log₁₀P of between 1 and 8, for example, those set forth in the following Table II:

TABLE II

| | Clog₁₀P | Molecular |
|---|---|---|
| Fragance Component | value | Weight |
| benzaldehyde | 1.480 | 106.12 |
| benzyl acetate | 1.960 | 150.17 |
| laevo-carvone | 2.083 | 150.22 |
| geraniol | 2.649 | 154.26 |
| cis-jasmone | 2.712 | 164.25 |
| β-phenylethyl alcohol | 1.183 | 122.17 |
| α-terpineol | 2.569 | 154.25 |
| δ-nonalactone | 2.760 | 156.23 |
| 1-phenyl hexanol-5 | 3.299 | 178.28 |
| dihydromyrcenol | 3.03 | 156.27 |
| δ-undecalactone | 3.830 | 184.28 |
| amyl cinnamate | 3.771 | 218.30 |
| benzophenone | 3.120 | 182.22 |
| nerol | 2.649 | 154.25 |
| 2-methoxynaphthalene | 3.235 | 158.20 |
| ethyl undecylenate | 4.888 | 212.34 |
| geranyl anthranilate | 4.216 | 273.38 |
| α-irone | 3.820 | 206.33 |
| α-santalol | 3.800 | 220.36 |
| iso-eugenol | 2.547 | 164.21 |
| amyl salicylate | 4.601 | 208.26 |
| benzyl salicylate | 4.383 | 228.25 |
| β-caryophyllene | 6.333 | 204.36 |
| cedrol | 4.530 | 222.37 |
| cedryl acetate | 5.436 | 264.41 |
| cedryl formate | 5.070 | 238.37 |
| cyclohexyl salicylate | 5.265 | 220.29 |
| γ-dodecalactone | 4.359 | 198.31 |
| ethyl undecylenate | 4.888 | 212.34 |
| geranyl anthranilate | 4.216 | 273.38 |
| β-phenylethyl benzoate | 4.058 | 226.38 |
| β-phenylethyl phenyl acetate | 3.767 | 240.31 |
| 5-acetyl-1,1,2,3,3,6-hexamethyl | 5.977 | 258.41 |
| indane | | |
| cyclopentadecanolide | 6.246 | 240.39 |
| d-limonene | 4.232 | 136.24 |
| cis-p-t-butylcyclohexyl acetate | 4.019 | 198.31 |
| amyl cinnamic aldehyde | 4.324 | 202.30 |

It is to be understood that the preferred range of concentration of functional product contained in the slurry of our invention, both encapsulated and non-confined is from about 0.03 to about 0.8% by weight; preferably from about 0.05 to about 0.3 percent by weight of the slurry.

Subsequent to the slurry being applied to the fabric article according to the process of our invention, the algorithm of the scaled intensity of functional product evolved from the thus-treated article, Y, vs. time (in days), X is as follows:
Y=αX³+βX²+γX+δ
wherein
−0.05≦α≦+0.03;
−2.0≦β≦+0.3;
−1.0≦γ≦+5.0; and
−1.0≦δ≦+5.0.

### BRIEF DESCRIPTION OF THE DRAWINGS

FIGS. 1, 2, 3 and 4 are sets of bar graphs for, respectively, 0, 3, 7 and 14 day evaluation periods comparing fragrance (of Example A described herein) intensity on a scale of 0-5 (measured on the “Y” axis) vs. time (measured on the “X” axis) for (a) fragrances evolving from several types of microcapsule compositions, for example, those containing microcapsules composed of melamine formaldehyde polymeric shells containing functional product, those composed of urea formaldehyde shells containing functional product, those composed of melamine-formaldehyde shells containing functional product with the slurry having added thereto silicone phospholipid polymers in a concentration of 0.2% and those composed of microcapsules formed by coacervation of a gelatin capsule around a liquid phase fragrance composition and (b) neat fragrance, each being deposited in an amount of 1 gram on circular areas of 1″ radius of wearable fabric articles (cotton T-shirts) using the process of our invention both before rubbing and after rubbing. In all cases, the fragrance in the composition being evaluated is at a level of 0.05% by weight of the composition being evaluated. In all cases each of the microcapsules and the neat fragrance is contained in a base consisting of water, ethanol, suspending agent, non-ionic surfactant, preservative and silicone polymer.

The evaluation at “0” time is herein intended to mean an evaluation taking place 3 hours subsequent to the composition application onto the fabric article, in order that the composition is dry at the time of evaluation.

FIG. 5 is a set of 10 day stability bar graphs for an initial evaluation (3 hours after application) comparing, at room temperature (68° F.) and at oven temperature (120° F.), fragrance intensity on a scale of 0-5 (measured on the “Y” axis) vs. time (measured on the “X” axis) for (a) fragrances evolving from several types of microcapsules, such as those composed of melamine formaldehyde polymeric shells, those composed of urea formaldehyde shells, and those formed by coacervation of a gelatin capsule around a liquid phase fragrance composition (powdered and non-powdered) and (b) neat fragrance deposited on fabric swatches using the process of our invention both before rubbing and after rubbing. In all cases each of the microcapsules and the neat fragrance is contained in a base consisting of water, ethanol, suspending agent, non-ionic surfactant, preservative and silicone polymer. In all cases, the fragrance in the composition being evaluated is at a level of 0.05% by weight of the composition.

FIG. 6 is a set of stability bar graphs for an initial head space analysis (via total area count in a gas chromatogram) and a 10 day head space analysis (via total area count in a gas chromatogram) comparing, at refrigerator temperature (38° F.), at room temperature (68° F.) and at oven temperature (120° F.), headspace fragrance concentration as measured by gas chromatogram area count (on the “Y” axis) for a base consisting of water, ethanol, suspending agent, non-ionic surfactant, preservative and silicone polymer containing (a) no fragrance, (b) neat fragrance or (c) fragrance microencapsulated in microcapsules composed of melamine-formaldehyde polymeric shells deposited on fabric swatches using the process of our invention. Measurements are carried out before rubbing.

FIGS. 7A, 7B, 7C and 7D are sets of bar graphs for, respectively, 1, 2, 3 and 4 day evaluation periods comparing fragrance (of Example B, infra) intensity on a scale of 0-5 (measured on the “Y” axis) vs. time (measured on the “X” axis) for (a) fragrance evolving from microcapsules composed of melamine formaldehyde polymeric shells; (b) neat fragrance (of Example B, infra); (c) both fragrance evolving from microcapsules composed of melamine formaldehyde polymeric shells (50%) and neat fragrance (of Example B, infra) (50%); and (d) commercial base (Stop and Shop, Inc. FABRIC EASE base) each deposited on a circular area of 1″ radius of a fabric article (cotton T-shirts) in an amount of 1 gram using the process of our invention. Evaluations are carried out both before rubbing and after rubbing.

In all cases except for the one concerning the commercial base, each of the microcapsules, and the neat fragrance is contained in a base consisting of water, ethanol, suspending agent, non-ionic surfactant, preservative and silicone polymer.

In all cases, the fragrance in the composition being evaluated is at a level of 0.10% by weight of the composition being evaluated.

In all cases, the evaluations are made by a 10 member expert panel.

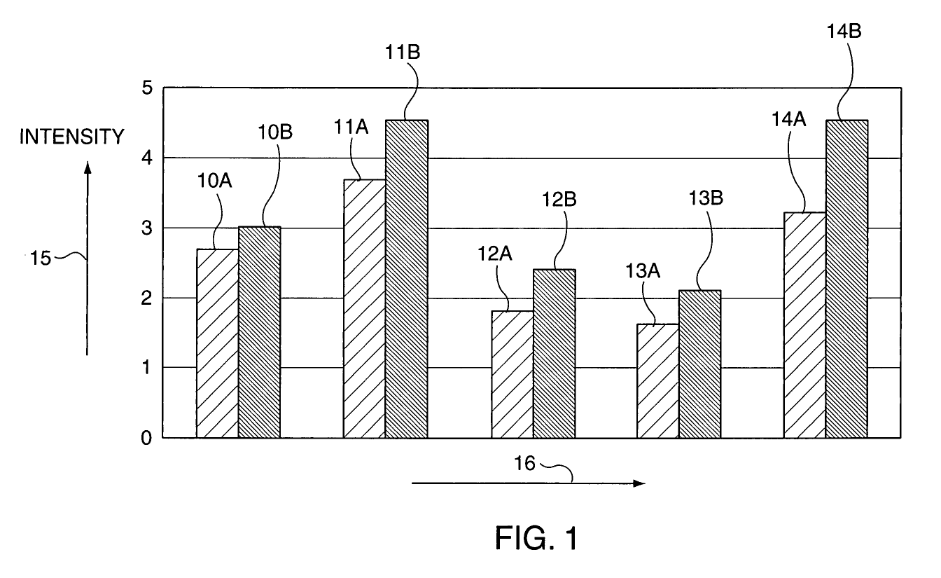

FIG. 8 is a set of graphs of mathematical models for evaluations of scaled intensity vs. time (in days) for each of the results of FIGS. 1, 2, 3 and 4. The evaluations are those of non-rubbed and rubbed fabric articles (cotton T-shirts) having applied thereto in an amount of 1 gram on a circular area 1″ in radius, slurries containing (a) melamine-formaldehyde polymeric fragrance-containing microcapsules and (b) slurries containing melamine-formaldehyde polymeric fragrance-containing microcapsules with 0.2% by weight of a silicone phospholipid polymer being added thereto. In each case the mathematical model is in accordance with the generic equation:
Y=αX³+βX²+γXαδ

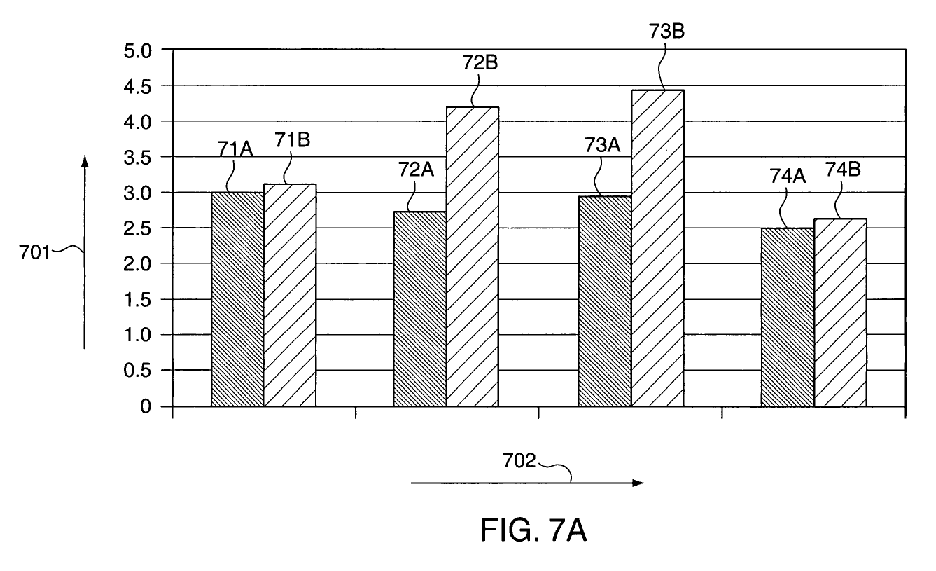

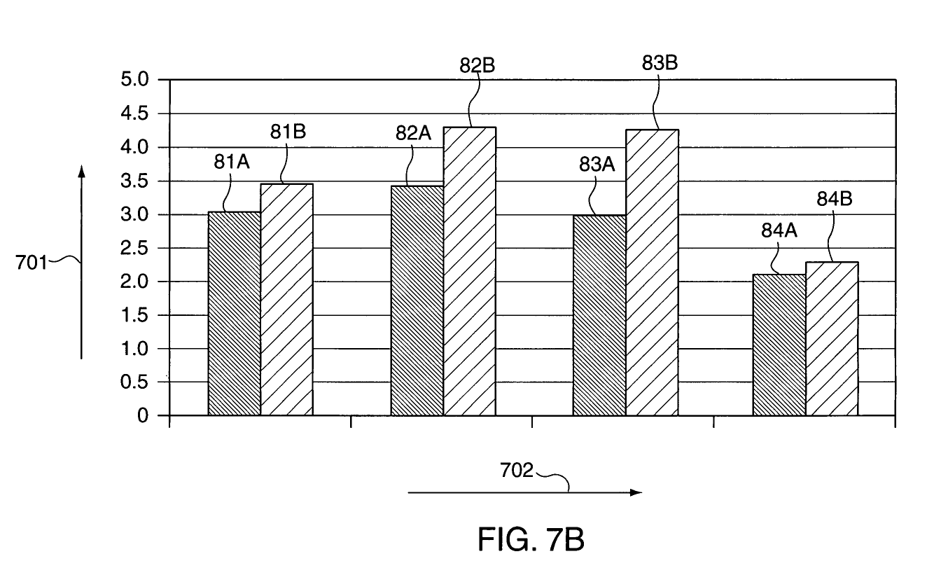

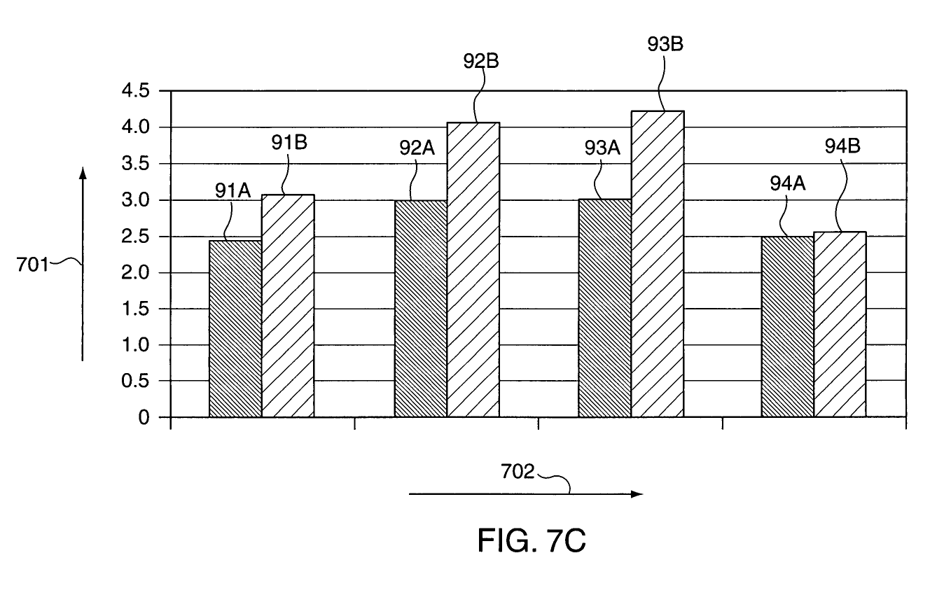

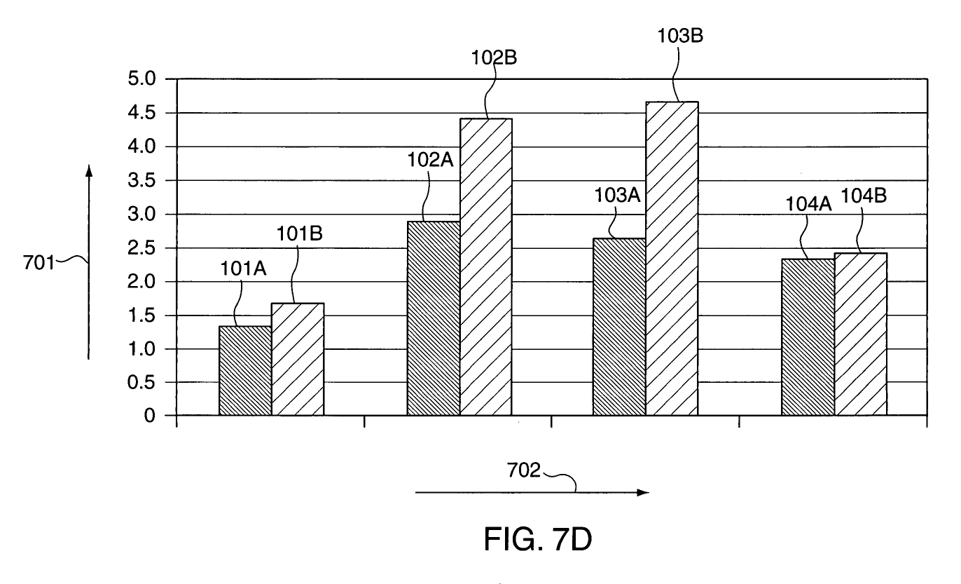

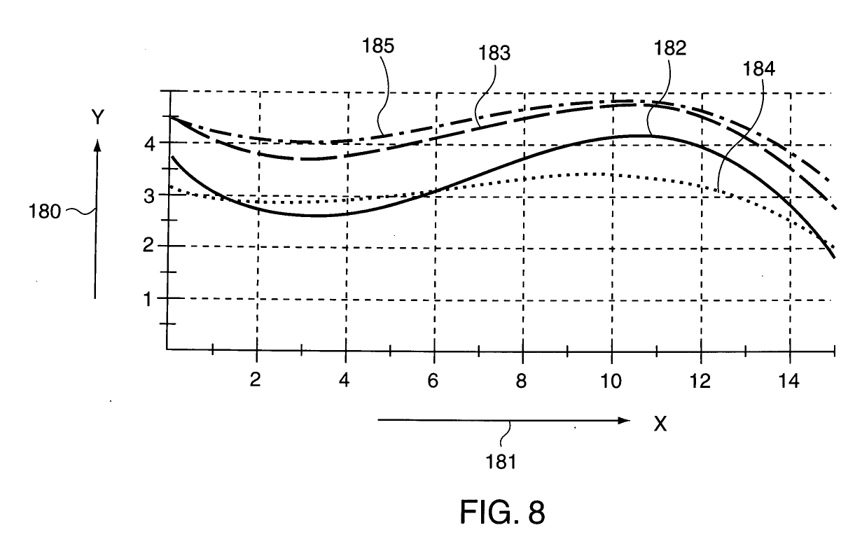

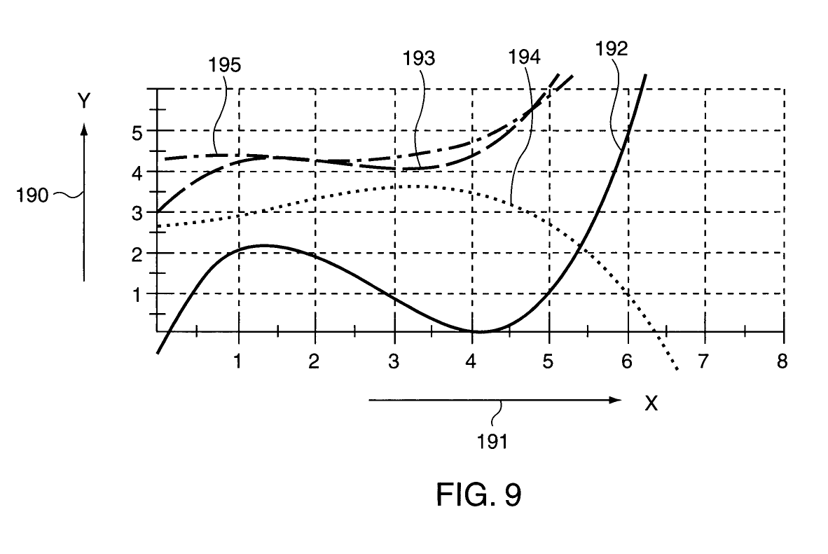

FIG. 9 is a set of graphs of mathematical models for evaluations of scaled intensity vs. time (in days) for each of the results of FIGS. 7A, 7B 7C and 7D. The evaluations are for non-rubbed and rubbed fabric articles (cotton T-shirts) having applied thereto in an amount of I gram on a circular area 1″ in radius, slurries containing (a) melamine-formaldehyde polymeric fragrance containing microcapsules and (b) 50:50 mixtures of non-encapsulated fragrance and melamine-formaldehyde polymeric fragrance containing microcapsules. In each case the mathematical model is in accordance with the generic equation:
Y=αX³+βX²+γX+δ

### DETAILED DESCRIPTION OF THE DRAWINGS

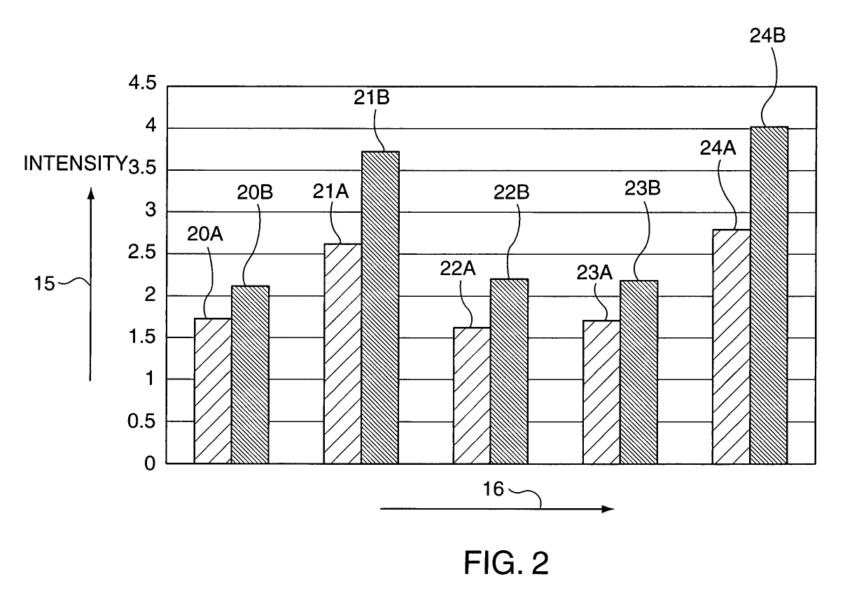

In FIGS. 1, 2, 3 and 4 the “X” axis, along which each of the bar graphs is placed, is indicated by reference numeral 16, and the “Y” axis, representing scaled intensity of fragrance (on a scale of (0-5) observed after treatment is indicated by reference numeral 15. FIG. 1 sets forth evaluations at 0 days (3 hours after application of the test substance to the fabric article). FIGS. 2, 3 and 4 respectively set forth evaluations at 3, 7 and 14 days. The bar graphs indicated by reference numerals 10A, 20A, 30A and 40A are for non-rubbed fabric articles (cotton T-shirts) having neat fragrance (of Example A infra) applied in an amount of 1 gram over a circular area of 1″ in radius, and their evaluations at, respectively, 0, 3, 7 and 14 days. The bar graphs indicated by reference numerals 10B, 20B, 30B and 40B are for rubbed fabric articles (cotton T-shirts) having neat fragrance (of Example A infra) applied in an amount of 1 gram over a circular area of 1″ in radius, and their evaluations at, respectively, 0, 3, 7 and 14 days. The bar graphs indicated by reference numerals 11A, 21A, 31A and 41A are for non-rubbed fabric articles (cotton T-shirts) having microcapsules composed of melamine-formaldehyde polymeric shells encapsulating fragrance (of Example A infra) applied in an amount of 1 gram over a circular area 1″ in radius, and their evaluations at, respectively, 0, 3, 7 and 14 days. The bar graphs indicated by reference numerals 11B, 21B, 31B and 41B are for rubbed fabric articles (cotton T-shirts) having microcapsules composed of melamine-formaldehyde polymeric shells encapsulating fragrance (of Example A infra) applied in an amount of 1 gram over a circular area 1″ in radius and their evaluations at, respectively, 0, 3, 7 and 14 days. The bar graphs indicated by reference numerals 12A, 22A, 32A and 42A are for non-rubbed fabric articles (cotton T-shirts) having microcapsules composed of coacervated gelatin shells encapsulating fragrance (of Example A infra) applied in an amount of 1 gram over a circular area 1″ in radius and their evaluations at, respectively, 0, 3, 7 and 14 days. The bar graphs indicated by reference numerals 12B, 22B, 32B and 42B are for rubbed fabric articles (cotton T-shirts) having microcapsules composed of coacervated gelatin shells encapsulating fragrance (of Example A infra) applied in an amount of 1 gram over a circular area 1″ in radius and their evaluations at, respectively, 0,. 3, 7 and 14 days. The coacervated gelatin microcapsules containing fragrances are prepared in accordance with the process described in U.S. Pat. No. 2,800,457 and more particularly in Example D at Column 19, lines 51-65 of U.S. Pat. No. 4,428,869. The bar graphs indicated by reference numerals 13A, 23A, 33A and 43A are for non-rubbed fabric articles (cotton T-shirts) having microcapsules composed of urea-formaldehyde polymeric shells encapsulating fragrance (of Example A infra) applied in an amount of 1 gram over a circular area 1″ in radius and their evaluations at, respectively, 0, 3, 7 and 14 days. The fragrance-containing microcapsules composed of urea-formaldehyde shells are prepared in accordance with the procedure of Example 2 at Column 8, lines 28-47 of U.S. Pat. No. 3,516,846. The bar graphs indicated by reference numerals 13B, 23B, 33B and 43B are for rubbed fabric articles 9 cotton T-shirts) having microcapsules composed of urea-formaldehyde polymeric shells encapsulating fragrance (of Example A infra) applied in an amount of 1 gram over a circular area of 1″ in radius and their evaluations at, respectively, 0, 3, 7 and 14 days. The bar graphs indicated by reference numerals 14A, 24A, 34A and 44A are for non-rubbed fabric articles (cotton T-shirts) having a slurry of fragrance (of Example A) containing microcapsules composed of melamine-formaldehyde polymeric shells in admixture with 0.2% by weight of added polymeric silicone phospholipid (prepared according to Example 56 at Column 12, line 7 of U.S. Pat. No. 5,237,035) applied in an amount of 1 gram over a circular area of 1″ in radius and their evaluations at, respectively, 0, 3, 7 and 14 days. The bar graphs indicated by reference numerals 14B, 24B, 34B and 44B are for rubbed fabric articles (cotton T-shirts) having microcapsules composed of melamine-formaldehyde polymeric shells in admixture with 0.2% by weight of added polymeric silicone phospholipid (prepared according to Example 56 at Column 12, line 7 of U.S. Pat. No. 5,237,035) encapsulating fragrance (of Example A infra) applied in an amount of 1 gram over a circular area of 1″ in radius and their evaluations at, respectively, 0, 3, 7 and 14 days.

The following table sets forth the weight percent fragrance, weight percent fragrance containing microcapsules, and weight percent fragrance-free microcapsules contained in the application slurries used in compiling the data for FIGS. 1, 2, 3 and 4, described above:

TABLE III

| | | % microcapsule | |
|---|---|---|---|
| | % | containing | % microcapsule (in |
| Bar Graph | Fragrance | fragrance in | absence of fragrance) |
| Group | in Slurry | slurry | in slurry |
| A | 0.05 | 0.00 | 0.00 |
| B | 0.05 | 0.18 | 0.13 |
| C | 0.05 | 0.21 | 0.16 |
| D | 0.05 | 0.07 | 0.02 |
| E | 0.05 | 0.38 | 0.33 |

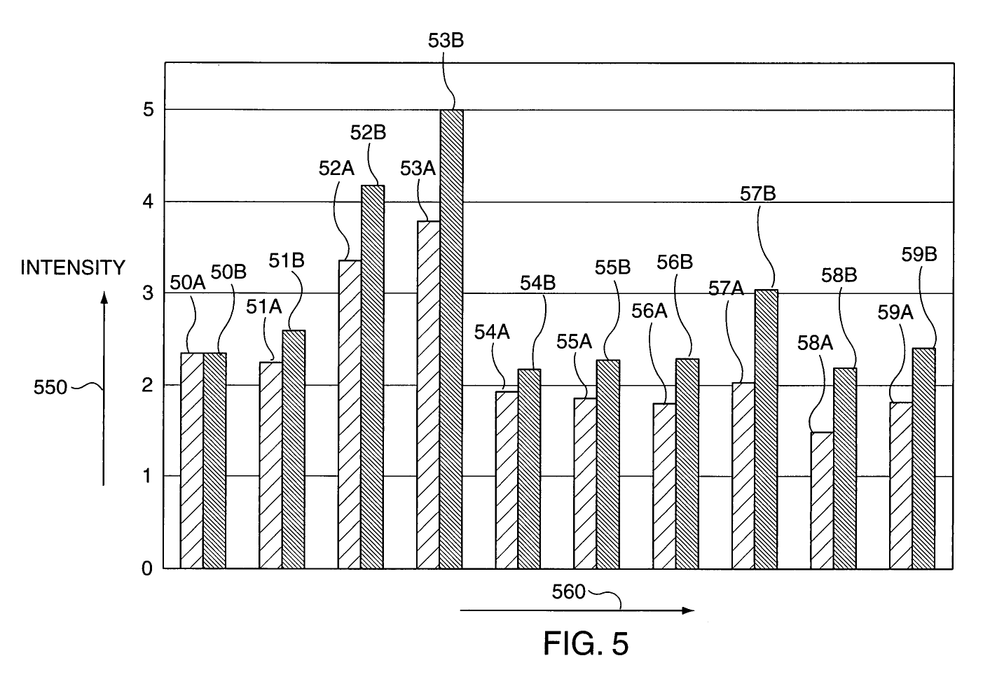

In FIG. 5 showing the results of 10 day stability studies, the “X” axis, along which each of the bar graphs is placed, is indicated by reference numeral 560, and the “Y” axis, representing scaled intensity of fragrance (on a scale of 0-5) observed after treatment is indicated by reference numeral 550. The bar graphs indicated by reference numerals 50A and 51A are for the evaluations of non-rubbed fabric swatches maintained, respectively, at 120° F. and 68° F., having neat fragrance (of Example A infra) applied. The bar graphs indicated by reference numerals 50B and 51B are for the evaluations of rubbed fabric swatches maintained, respectively at 120° F. and 68° F., having neat fragrance (of Example A infra) applied. The bar graphs indicated by reference numerals 52A and 53A are for the evaluations of non-rubbed fabric swatches maintained, respectively, at 120° F. and 68° F., having microcapsules composed of melamine-formaldehyde polymeric shells encapsulating fragrance (of Example A infra) applied. The bar graphs indicated by reference numerals 52B and 53B are for the evaluations of rubbed fabric swatches, maintained, respectively at 120° F. and 68° F., having microcapsules composed of melamine-formaldehyde polymeric shells encapsulating fragrance (of Example A infra) applied. The bar graphs indicated by reference numerals 54A and 55A are for the evaluations of non-rubbed fabric swatches, maintained respectively at 120° F. and 68° F., having microcapsules composed of coacervated gelatin shells encapsulating fragrance (of Example A infra) applied. The bar graphs indicated by reference numerals 54B and 55B are for the evaluations of rubbed fabric swatches, maintained respectively at 120° F. and 68° F., having microcapsules composed of coacervated gelatin shells encapsulating fragrance (of Example A infra) applied. The coacervated gelatin microcapsules containing fragrances are prepared in accordance with the process described in U.S. Pat. No. 2,800,457 and more particularly in Example D at Column 19, lines 51-65 of U.S. Pat. No. 4,428,869. The bar graphs indicated by reference numerals 56A and 57A are for the evaluations of non-rubbed fabric swatches, maintained respectively at 120° F. and 68° F., having microcapsules composed of urea-formaldehyde polymeric shells encapsulating fragrance (of Example A infra) applied. The bar graphs indicated by reference numerals 56B and 57B are for the evaluations of rubbed fabric swatches, maintained respectively at 120° F. and 68° F., having microcapsules composed of urea-formaldehyde polymeric shells encapsulating fragrance (of Example A infra) applied. The bar graphs indicated by reference numerals 58A and 59A are for the evaluations of non-rubbed fabric swatches, maintained respectively at 120° F. and 68° F., having powdered microcapsules composed of coacervated gelatin shells encapsulating fragrance (of Example A infra) applied. The bar graphs indicated by reference numerals 58B and 59B are for the evaluations of rubbed fabric swatches, maintained respectively at 120° F. and 68° F., having powdered microcapsules composed of coacervated gelatin shells encapsulating fragrance (of Example A infra) applied.

In all cases, the fragrance in the composition being evaluated is at a level of 0.05% by weight of the composition being evaluated.

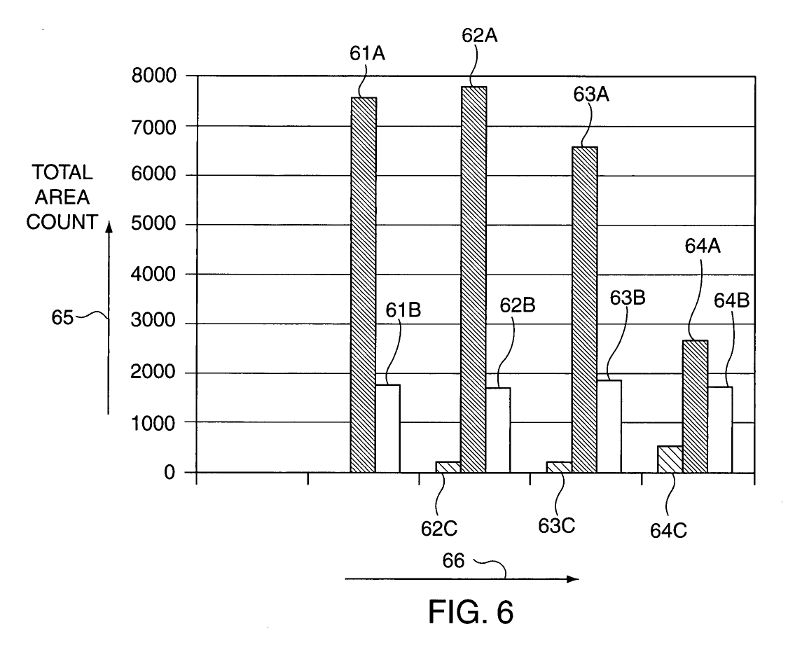

In FIG. 6, the set of stability bar graphs, total area count is measured on the “Y” axis, indicated by reference numeral 65 and the bar graphs are indicated along the “X” axis, indicated by reference numeral 66. The bar graphs indicated by reference numerals 61A, 62A, 63A and 64A are, respectively, for the measurement of the stability of neat fragrance (of Example A infra) at a level of 0.1% in base containing water, ethanol, suspending agent, non-ionic surfactant, preservative and silicone polymer for 0 days, 10 days at refrigeration temperature (38° F.), 10 days at room temperature (68° F.) and 10 days at oven temperature (120° F.). The bar graphs indicated by reference numerals 61B, 62B, 63B and 64B are, respectively, for the measurement of the stability of microcapsules composed of melamine-formaldehyde polymeric shells encapsulating fragrance (of Example A infra) at a fragrance level of 0.1% in base containing water, ethanol, suspending agent, non-ionic surfactant, preservative and silicone polymer for 0 days, 10 days at refrigeration temperature (38° F.), 10 days at room temperature (68° F.) and 10 days at oven temperature (120° F.). The bar graphs indicated by reference numerals 62C, 63C and 64C are, respectively, for the measurement of the stability of microcapsules composed of empty melamine-formaldehyde polymeric shells (not containing any fragrance or other functional product) for 10 days at refrigeration temperature (38° F.), 10 days at room temperature (68° F.) and 10 days at oven temperature (120° F.). The weight % microcapsule containing fragrance in the slurry is 0.27% and the weight % empty microcapsule in the slurry is 0.17%. The results shown in FIG. 6 are indicative of the high degree of stability of fragrances encapsulated in microcapsules composed of melamine-formaldehyde polymeric shells.

In FIGS. 7A, 7B, 7C and 7D the “X” axis, along which each of the bar graphs is placed, is indicated by reference numeral 702 and the “Y” axis, representing scaled intensity of fragrance (on a scale of (0-5) observed after treatment is indicated by reference numeral 701.

In FIG. 7A, showing test results at 24 hours (I day) after application, the bar graphs indicated by reference numerals 71A, 72A, 73A and 74A represent the evaluations of non-rubbed fabric articles (cotton T-shirts) respectively having applied thereto in an amount of 1 gram over a circular area 1″ in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base consisting of water, ethanol, suspending agent, non-ionic surfactant, preservative and silicone polymer and 50% unconfined (“non-encapsulated”) fragrance (of Example B, infra) at a level of 0.05% in a base consisting of water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base consisting of water, ethanol, suspending agent, non-ionic surfactant, preservative and silicone polymer and (d) a commercial base (Stop and Shop, Inc. FABRIC EASE base); and the bar graphs indicated by reference numerals 71B, 72B, 73B and 74B represent evaluations of rubbed fabric articles (cotton T-shirts) respectively having applied thereto in an amount of 1 gram over a circular area 1″ in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base containing water, ethanol, suspending agent, non-ionic surfactant, preservative and silicone polymer and 50% non-encapsulated fragrance (of Example B, infra) at a level of 0.05% in a base containing water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base consisting of water, ethanol, suspending agent, non-ionic surfactant, preservative and silicone polymer and (d) a commercial base (Stop and Shop, Inc. FABRIC EASE base).

In FIG. 7B, showing test results at 48 hours (2 days) after application, the bar graphs indicated by reference numerals 81A, 82A, 83A and 84A represent evaluations of non-rubbed fabric articles (cotton T-shirts) respectively having applied thereto in an amount of 1 gram over a circular area 1″ in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base consisting of water, ethanol, suspending agent, non-ionic surfactant, preservative and silicone polymer and 50% unconfined (“non-encapsulated”) fragrance (of Example B, infra) at a level of 0.05% in a base consisting of water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base consisting of water, ethanol, suspending agent, non-ionic surfactant, preservative and silicone polymer and (d) a commercial base (Stop and Shop, Inc. FABRIC EASE base); and the bar graphs indicated by reference numerals 81B, 82B, 83B and 84B represent evaluations of rubbed fabric articles (cotton T-shirts) respectively having applied thereto in an amount of 1 gram over a circular area 1″ in radius (a) neat fragrance (of Example B, infra) contained at a level of 0.1% in a base containing water, ethanol; suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base containing water, ethanol, suspending agent, non-ionic surfactant, preservative and silicone polymer and 50% non-encapsulated fragrance (of Example B, infra) at a level of 0.05% in a base containing water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base containing water, ethanol, suspending agent, non-ionic surfactant, preservative and silicone polymer and (d) a commercial base (Stop and Shop, Inc. FABRIC EASE base).

In FIG. 7C, showing test results at 72 hours (3 days) after application, the bar graphs indicated by reference numerals 91A, 92A, 93A and 94A represent evaluations of non-rubbed fabric articles (cotton T-shirts) respectively having applied thereto in an amount of 1 gram over a circular area of 1″ in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base consisting of water, ethanol, suspending agent, non-ionic surfactant, preservative and silicone polymer and 50% non-encapsulated fragrance (of Example B, infra) at a level of 0.05% in a base consisting of water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base-consisting of water, ethanol, suspending agent, non-ionic surfactant, preservative and silicone polymer and (d) a commercial base (Stop and Shop, Inc. FABRIC EASE base); and the bar graphs indicated by reference numerals 91B, 92B, 93B and 94B represent evaluations of rubbed fabric articles (cotton T-shirts) respectively having applied thereto in an amount of I gram over a circular area 1″ in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base consisting of water, ethanol, suspending agent, non-ionic surfactant, preservative and silicone polymer and 50% non-encapsulated fragrance (of Example B, infra) at a level of 0.05% in a base consisting of water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base consisting of water, ethanol, suspending agent, non-ionic surfactant, preservative and silicone polymer and (d) a commercial base (Stop and Shop, Inc. FABRIC EASE base).

In FIG. 7D, showing test results at 96 hours (4 days) after application, the bar graphs indicated by reference numerals 101A, 102A, 103A and 104A represent evaluations of non-rubbed fabric articles (cotton T-shirts) respectively having applied thereto in an amount of 1 gram over a circular area 1″ in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base consisting of water, ethanol, suspending agent, non-ionic surfactant, preservative and silicone polymer consisting of water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base consisting of water, ethanol, suspending agent, non-ionic surfactant, preservative and silicone polymer and (d) a commercial base (Stop and Shop, Inc. FABRIC EASE base); and the bar graphs indicated by reference numerals 101B, 102B, 103B and 104B represent evaluations of rubbed fabric articles (cotton T-shirts) respectively having applied thereto in an amount of 1 gram over a circular area 1″in radius (a) neat fragrance (of Example B, infra) contained at a level of 0.1% in a base consisting of water, ethanol, suspending agent, non-ionic surfactant, preservative and silicone polymer; (b) a composition consisting of 50% microencapsulated fragrance (of Example B, infra) contained in microcapsules composed of melamine-formaldehyde shells wherein the fragrance is contained at a level of 0.05% in a base consisting of water, ethanol, suspending agent, non-ionic surfactant, preservative and silicone polymer and 50% non-encapsulated fragrance (of Example B, infra) at a level of 0.05% in a base consisting of water, ethanol, suspending agent, non-ionic surfactant, preservative and silicone polymer; (c) a composition consisting of microencapsulated fragrance (of Example B, infra) contained in microcapsules composed of melamine-formaldehyde shells wherein the fragrance is contained at a level of 0.10% in a base consisting of water, ethanol, suspending agent, non-ionic surfactant, preservative and silicone polymer and (d) a commercial base (Stop and Shop, Inc. FABRIC EASE base).

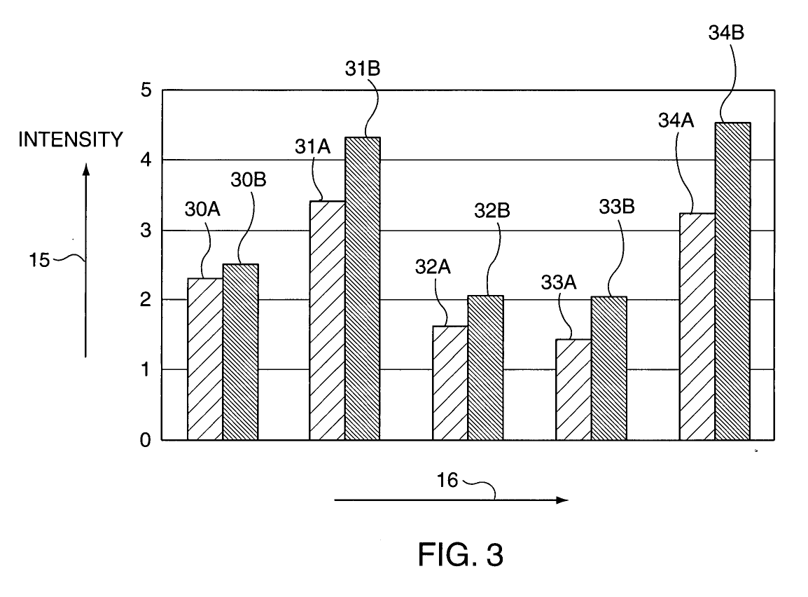

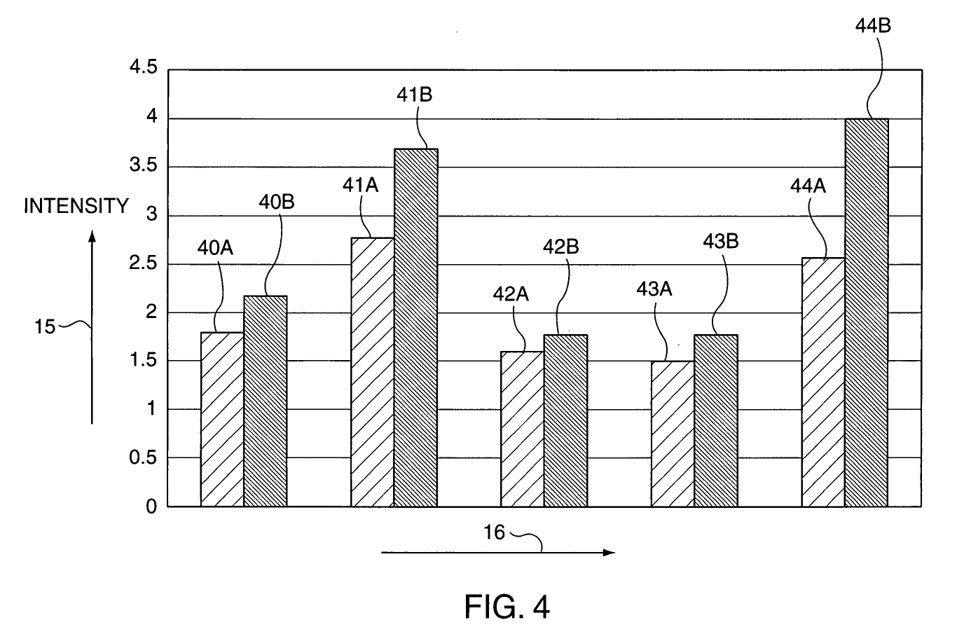

FIG. 8 shows a series of graphs of scaled intensity (on a scale of 0-5) vs. time (days) for the data shown in bar graphs 11A, 11B, 14A and 14B in FIG. 1; bar graphs 21A, 21B, 24A and 24B in FIG. 2; bar graphs 31A, 31B, 34A and 34B in FIG. 3 and bar graphs 41A, 41B, 44A and 44B in FIG. 4. Each of the graphs is a third degree equation. The scaled intensity is measured on the “Y” axis which is indicated by reference numeral 180. The time (in days) is measured along the “X” axis which is indicated by reference numeral 181. The following table sets forth the equation for each graph, the reference numeral indicating the graph, and the reference numerals identifying the specific bar graphs on which each graph is based:

TABLE IV

| Rubbed or unrubbed | Reference Numerals | | Reference |
|---|---|---|---|
| fabric article and | Identifying | | Numeral |
| substance applied thereto | Relevant Bar Graphs | Equation | for graph |
| Fragrances microencapsulated | 11A, 21A, | Y = −.0076X³ + 0.157X² − | 182 |
| in melamine-formaldehyde | 31A and | 0.770X + 3.7 | |
| polymeric microcapsules | 41A | | |
| (non-rubbed) | | | |
| Fragrances microencapsulated | 11B, 21B, | Y = −.0058X³ + 0.117X² − | 183 |
| in melamine-formaldehyde | 31B and | 0.567X + 4.5 | |
| polymeric microcapsules (rubbed) | 41B | | |
| Fragrances microencapsulated | 14A, 24A, | Y = −.0036X³ + 0.069X² − | 184 |
| in melamine-formaldehyde | 34A and | 0.309X + 3.2 | |
| polymeric microcapsules | 44A | | |
| which are in admixture with | | | |
| polymeric silicone phospho- | | | |
| lipid additive (non-rubbed) | | | |
| Fragrances microencapsulated | 14B, 24B, | Y = −.0043X³ + 0.084X² − | 185 |
| in melamine-formaldehyde | 34B and | 0.381X + 4.5 | |
| polymeric microcapsules | 44B | | |
| which are in admixture with | | | |
| polymeric silicone phospho- | | | |
| lipid additive (rubbed) | | | |

FIG. 9 shows a series of graphs of scaled intensity (on a scale of 0-5) vs. time (days) for the data shown in bar graphs 72A, 72B, 73A and 73B in FIG. 7A; bar graphs 82A, 82B, 83A and 83B in FIG. 7B; bar graphs 92A, 92B 93A and 93B in FIG. 7C and bar graphs 102A, 102B, 103A and 103B in FIG. 7D. Each of the graphs is a third degree equation. The scaled intensity is measured on the “Y” axis which is indicated by reference numeral 190. The time (in days) is measured along the “X” axis which is indicated by reference numeral 191. The following table sets forth the equation for each graph, the reference numeral indicating the graph, and the reference numerals identifying the specific bar graphs on which each graph is based:

TABLE V

| Rubbed or unrubbed | Reference Numerals | | Reference |
|---|---|---|---|
| fabric article and | Identifying | | Numeral |
| substance applied thereto | Relevant Bar Graphs | Equation | for graph |
| 50:50 Mixture of | 72A, 82A, | Y = 0.233X³ − 1.95X² + 4.197X − 0.5 | 192 |
| fragrances + fragrances | 92A and | | |
| microencapsulated in | 102A | | |
| melamine-formaldehyde | | | |
| polymeric microcapsules | | | |
| (non-rubbed) | | | |
| 50:50 Mixture of | 72B, 82B, | Y = 0.133X³ − 0.95X² + 2.017X + 3.0 | 183 |
| fragrances + fragrances | 92B and | | |
| microencapsulated in | 102B | | |
| melamine-formaldehyde | | | |
| polymeric microcapsules | | | |
| (rubbed) | | | |
| Fragrances microencapsulated | 73A, 83A, | Y = − .05X³ + 0.25X² − 0.3X + 4.3 | 194 |
| in melamine-formaldehyde | 93A and | | |
| polymeric microcapsules | 93A | | |
| (non-rubbed) | | | |
| Fragrances microencapsulated | 73B, 83B, | Y = 0.05X³ − 0.25X² + 0.3X + 4.3 | 195 |
| in melamine-formaldehyde | 93B and | | |
| polymeric microcapsules | 103B | | |
| (rubbed) | | | |

The following examples are not meant to define or otherwise limit the scope of the invention. Rather the scope of the invention is to be ascertained according to the claims that follow the examples. Unless noted to the contrary, all percentages are given on a weight percent on a dry basis.

### EXAMPLE A

The following fragrance composition was prepared:

| | C log₁₀P | Molecular | Parts by |
|---|---|---|---|
| Fragrance Component | value | Weight | Weight |
| ethyl undecylenate | 4.888 | 212.34 | 3.0 |
| geranyl anthranilate | 4.216 | 273.38 | 7.5 |
| α-irone | 3.820 | 206.33 | 6.3 |
| phenyl ethyl benzoate | 4.058 | 226.28 | 3.2 |
| d-limonene | 4.232 | 136.24 | 3.2 |
| cis-p-t-butylcyclohexyl | 4.019 | 198.31 | 5.8 |
| acetate | | | |
| amyl cinnamic aldehyde | 4.324 | 202.30 | 7.3 |
| hexyl cinnamic aldehyde | 5.473 | 216.33 | 12.6 |
| hexyl salicylate | 5.260 | 222.29 | 12.6 |

### EXAMPLE B

The following fragrance was prepared:

| | C log₁₀P | Molecular | Parts by |
|---|---|---|---|
| Fragrance Component | value | Weight | Weight |
| ethyl undecylenate | 4.888 | 212.34 | 10.5 |
| geranyl anthranilate | 4.216 | 273.38 | 35.4 |
| α-irone | 3.820 | 206.33 | 5.3 |
| phenyl ethyl benzoate | 4.058 | 226.28 | 5.3 |
| phenylethyl phenyl acetate | 3.767 | 240.31 | 5.3 |
| 5-acetyl-1,1,2,3,3,6- | 5.977 | 258.41 | 2.5 |
| hexamethyl indane | | | |
| cyclopentadecanolide | 6.246 | 240.39 | 7.5 |
| d-limonene | 4.232 | 136.24 | 25.0 |
| cis-p-t-butylcyclohexyl | 4.019 | 198.31 | 4.0 |
| acetate | | | |
| amyl cinnamic aldehyde | 4.324 | 202.30 | 4.0 |

### EXAMPLE 1

At the following rate the fragrance of Example A was microencapsulated into the following microcapsules in accordance with the processes stated:

TABLE VI

| | Reference to Process | % of | |
|---|---|---|---|
| | for formation of | functional product | |
| | microencapsulated | of Example A | Sample |
| Nature of Capsule | functional product | in microcapsule | Identification |
| Melamine-formaldehyde | U.S. Pat. Nos. 3,516,846 | 27.8% | B |
| polymer shell having | and 6,413,548 | | |
| average effective diameter | | | |
| of 5-10 microns | | | |
| Coacervated gelatin shell | U.S. Pat. No. 2,800,457 | 23.8% | C |
| having average effective | | | |
| diameter of 10-20 microns | | | |
| Urea-formaldehyde | U.S. Pat. No. 3,516,846 | 71.4% | D |
| polymer shell having | | | |
| average effective diameter | | | |
| of 5-10 microns | | | |
| Melamine-formaldehyde | U.S. Pat. Nos. 3,516,846; | 37.6% | E |
| polymer shell having | 6,413,548 and 5,237,035 | | |
| average effective diameter | | | |
| of 5-10 microns coated | | | |
| with 53% silicone phospho- | | | |
| lipid polymer | | | |

A sufficient amount of each of the microcapsule products in the above table was placed in the following base:
(a) water-90.7%
(b) ethanol-5%
(c) SURCIDE P (Preservative)—0.1%
(d) TOMADOL 91-8 (non-ionic surfactant)-2.7%
(e) Xanthan Gum-0.5%
(f) dimethyl silicone polymer-1.0%
   in order to cause the functional product to be 0.05% of the slurry in each case.

In each case, the microencapsulated functional product was suspended as a “suspended slurry”. 150 cubic centermeters of the resulting suspended slurry was then placed in a trigger sprayer as disclosed in U.S. Pat. No. 4,819,835. In addition, neat fragrance of Example A (non-confined) was placed in a fifth sample of the above-identified base at the rate of 0.05% (identified as sample A).

In an individual amount of 1 gram, each of the resulting products was then simultaneously sprayed onto circular areas, 1 inch in radius of five separate equally malodourous cotton T-shirts having identical tobacco malodours.

The results of the spraying are set forth on FIGS. 1, 2, 3, and 4 described above. The numerical results for scaled intensity on a scale of 0-5 are set forth in the following Table VII:

TABLE VII

| | 0 DAYS | | 3 DAYS | | 7 DAYS | | 14 DAYS | |
|---|---|---|---|---|---|---|---|---|
| Sample | not | | not | | not | | not | |
| Identification | rubbed | rubbed | rubbed | rubbed | rubbed | rubbed | rubbed | rubbed |
| A | 2.7 | 3 | 1.7 | 2.1 | 2.3 | 2.5 | 1.8 | 2.2 |
| B | 3.7 | 4.5 | 2.6 | 3.7 | 3.4 | 4.3 | 2.8 | 3.7 |
| C | 1.8 | 2.4 | 1.6 | 2.3 | 1.6 | 2.0 | 1.6 | 1.8 |
| D | 1.6 | 2.1 | 1.7 | 2.2 | 1.4 | 2.0 | 1.5 | 1.8 |
| E | 3.2 | 4.5 | 2.8 | 4.0 | 3.2 | 4.5 | 2.6 | 4.0 |

All evaluations leading to the results in Table VII were carried out by a 10 member expert panel.

The use of samples B and E, which both contained melamine-formaldehyde microencapsulated functional product covered the tobacco malodour in a manner far superior to that of samples A, C and D. In addition, the intensity of aesthetically-pleasing aroma prior to and after rubbing was significantly greater when using samples B and E than that of any aesthetically-pleasing aromas generated as a result of using any of samples A, C or D thereby showing the clear superiority of slurries containing microencapsulated functional products where the microcapsules are composed of melamine-formaldehyde polymeric shells.

### EXAMPLE 11

At the rate of 28% the fragrance of Example B was microencapsulated in microcapsules produced according to U.S. Pat. Nos. 3,516,846 and 6,413,548 and composed of melamine-formaldehyde polymer shells each of which had an average effective diameter of 5-10 microns.

A base was formulated containing the following ingredients:
(a) water—90.7%
(b) ethanol—5%
(c) SURCIDE P (Preservative)—0.1%
(d) TOMADOL 91-8 (non-ionic surfactant)—2.7%
(e) Xanthan Gum—0.5%
(f) dimethyl silicone polymer—1.0%

A first group of microcapsules was formed into a slurry with the above-mentioned base whereby the resulting slurry contained 0.1% by weight of fragrance and the weight % of microcapsules containing fragrance was 0.27%. The slurry was identified by the letter “γ”.

A second group of microcapsules was formed into a slurry with the above-mentioned base, in combination with non-confined fragrance of Example B whereby the resulting slurry contained 0.05% by weight of encapsulated fragrance and 0.05% by weight of non-confined (“non-encapsulated”) fragrance and the weight % of microcapsules containing fragrance was 0.135%. The slurry was identified by the letter “β”.

Non-confined fragrance of Example B as admixed with the above-mentioned base whereby the resulting formulation contained 0.1% by weight of fragrance. The resulting composition was identified by the letter “α”.

A fourth sample was a commercial base. FABRIC EASE, marketed by Stop & Shop, Inc. The commercial base was identified by the letter, “δ”.

Each of the compositions, “α”, “β”, “γ” and “δ” was applied via spraying in amounts of 1 gram each, separately, onto separate malodourous fabric articles (cotton T-shirts) emitting equal tobacco malodours, in circular areas having 1″ radii.

The results of the spraying are set forth in FIGS. 7A, 7B, 7C and 7d described herein. The numerical results for scaled intensity on a scale of 0-5 are set forth in the following Table VIII:

TABLE VIII

| | 1 DAY | | 2 DAYS | | 3 DAYS | | 4 DAYS | |
|---|---|---|---|---|---|---|---|---|
| Sample | not | | not | | not | | not | |
| Identification | rubbed | rubbed | rubbed | rubbed | rubbed | rubbed | rubbed | rubbed |
| α | 3.0 | 3.1 | 3.0 | 3.45 | 2.45 | 3.1 | 1.35 | 1.7 |
| β | 2.7 | 4.2 | 3.4 | 4.3 | 3.0 | 4.1 | 2.9 | 4.4 |
| γ | 2.9 | 4.4 | 3.0 | 4.3 | 3.0 | 4.3 | 2.6 | 4.7 |
| δ | 2.5 | 2.6 | 2.15 | 2.35 | 2.50 | 2.55 | 2.40 | 2.45 |

All evaluations leading to the results in Table VIII were carried out by a 10 member expert panel.

The use of samples “β” and “γ” which both contained melamine-formaldehyde microencapsulated functional product covered the tobacco malodour in a manner far superior to that of samples “α” and “δ”. In addition, the intensity of aesthetically-pleasing aroma prior to and after rubbing was significantly greater when using samples “β” and “γ” than that of any aesthetically-pleasing aromas generated as a result of using any of samples “α” or “δ” thereby showing the clear superiority of slurries containing microencapsulated functional products where the microcapsules are composed of melamine-formaldehyde polymeric shells over bases containing non-confined fragrances.

Each of the specifications and claims of each of the U.S. patents, patent applications and Published U.S. patent applications cited is herein incorporated by reference.

1. A method for substantively (a) imparting fragrance to and/or (b) substantially eliminating malodours from and/or (c) covering malodours evolved from and/or (d) preventing malodour formation in at least one fabric article for an extended period of time comprising the steps of:
i. providing one or more exposed surface areas of one or more fabric articles;
ii. providing an enclosure equipped with at least one pressure-activated air atomizer having an externally-located spray nozzle communicating with the interior of said enclosure, said nozzle having from 1 up to a plurality of nozzle exit ports having nozzle exit port effective diameters, D_{NPi};
iii. preparing a plurality of microcapsules each of which is composed of a rupturable external wall of a melamine-formaldehyde polymer enclosing from about 10 weight % to about 30 weight % of a first functional substance selected from the group consisting of (a) a fragrance composition each of the components of which has a C log₁₀P of between 2.5 and 8, wherein P is the n-octanol/water partition coefficient of said component; (b) a malodour counteracting composition each of the components of which has a C log₁₀P of between 2.5 and 8, wherein P is the n-octanol/water partition coefficient of said component; and (c) a malodour-preventing composition each of the components of which has a C log₁₀P of between 2.5 and 8, wherein P is the n-octanol/water partition coefficient of said component, each of said microcapsules having an average effective diameter \overline{D_{MC}} of from about 5 microns to about 80 microns, each of which microcapsule has an effective diameter of D_{MCi} wherein the smallest of D_{NPi} is substantially greater than the greatest of D_{MCi} wherein
$$\overline{{D}_{MC}} = \frac{1}{N} {ΣD}_{MCi}$$
and N represents the number of microcapsules in the slurry contained in said enclosure;
iv. providing an aqueous solution comprising (a) water, (b) a compatible solvent selected from the group consisting of ethanol, the mono-C₁ or C₂ ether of a mono-, di-, or tri-1,2-propylene glycol and the di-C₁ or C₂ ether of a mono-, di- or tri-1,2-propylene glycol, (c) a silicone polymer, (d) a compatible non-ionic surfactant, (e) a compatible preservative and (f) a compatible suspending agent;
v. admixing said plurality of microcapsules with said aqueous solution at a level of from about 0.1 weight % to about 0.4 weight % of microcapsules based o the weight of aqueous solution, thereby forming a microcapsule slurry wherein said microcapsules are suspended in said slurry and each of said microcapsules has a settling velocity in said slurry, Vs equal to about 0;
vi. optionally causing a non-encapsulated second functional substance selected from the group consisting of (a) a fragrance composition each of the components of which has a C log₁₀P of between 1 and 8, wherein P is the n-octanol/water partition coefficient of said component; (b) a malodour counteracting composition each of the components of which has a C log₁₀P of between 1 and 8, wherein P is the n-octanol/water partition coefficient of said component; and (c) a malodour-preventing composition each of the components of which has a C log₁₀P of between 1 and 8, wherein P is the n-octanol/water partition coefficient of said component to be in admixture with said slurry by means of admixing said second functional composition (A) with said aqueous solution or (B) with said slurry;
vii. placing said microcapsule slurry into said enclosure;
viii. situating said enclosure whereby the nozzle exit ports of the at least one externally-located spray nozzle are each substantially located in a plane substantially parallel to and opposite said one or more exposed surface areas of said one or more fabric articles at a substantially perpendicular mean distance of from about 0 to about 3 meters from said one or more exposed surface areas of said one or more fabric articles:
ix. applying sufficient pressure to said slurry located within said enclosure to enable said slurry to be sprayed through said one or more nozzle exit ports onto said one or more exposed surface areas of said one or more fabric articles whereby said microcapsules are effectively adhered to said one or more exposed surface areas of said one or more fabric articles thereby forming one or more microcapsule-fixed fabric article surface areas; and
whereby (a) the concentration of the functional substance contained in the slurry is from about 0.03% to about 0.8% immediately prior to the step ix and (b) subsequent to the step ix when said microcapsule-fixed fabric article surface areas are rubbed, said microcapsules rupture, thereby emitting said first functional substance.

2. The process of claim 1 wherein a non-encapsulated second functional substance selected from the group consisting of (a) a fragrance composition each of the components of which has a C log₁₀P of between 1 and 8, wherein P is the n-octanol/water partition coefficient of said component; (b) a malodour counteracting composition each of the components of which has a C log₁₀P of between 1 and 8, wherein P is the n-octanol/water partition coefficient of said component; and (c) a malodour-preventing composition each of the components of which has a C log₁₀P of between 1 and 8, wherein P is the n-octanol/water partition coefficient of said component is caused to be in admixture with said slurry by means of admixing said second functional composition (A) with said aqueous solution or (B) with said slurry.

3. The process of claim 1 wherein said first functional substance comprises a mixture of zinc ricinoleate or a solution thereof and a substituted monocyclic organic compound selected from the group consisting of:
1-cyclohexylethan-1-yl butyrate;
1-cyclohexylethan-1-yl acetate;
1-cyclohexylethan-1-ol;
1-(4′-methylethyl)cyclohexylethan-1-yl propionate; and
2′-hydroxy-1′-ethyl(2-phenoxy)acetate.

4. The process of claim 2 wherein said first functional substance and said second functional substance each comprises a mixture of zinc ricinoleate or a solution thereof and a substituted monocyclic organic compound selected from the group consisting of:
1-cyclohexylethan-1-yl butyrate;
1-cyclohexylethan-1-yl acetate;
1-cyclohexylethan-1-ol;
1-(4′-methylethyl)cyclohexylethan-1-yl propionate; and
2′-hydroxy-1′-ethyl(2-phenoxy)acetate.

5. The process of claim 1 wherein said first functional substance is a fragrance composition which effects malodour suppression.

6. The process of claim 2 wherein said first functional substance is a fragrance composition which effects malodour suppression and said second functional substance is a fragrance composition which effects malodour suppression.

7. The process of claim 2 wherein said first functional substance comprises a mixture of zinc ricinoleate or a solution thereof and a substituted monocyclic organic compound selected from the group consisting of:
1-cyclohexylethan-1-yl butyrate;
1-cyclohexylethan-1-yl acetate;
1-cyclohexylethan-1-ol:
1-(4′-methylethyl)cyclohexylethan-1-yl propionate; and
2-hydroxy-1′-ethyl(2-phenoxy)acetate
and said second functional substance is a fragrance composition which effects malodour suppression.

8. The process of claim 2 wherein said first functional substance is a fragrance composition which effects malodour suppression and said second functional substance comprises a mixture of zinc ricinoleate or a solution thereof and a substituted monocyclic organic compound selected from the group consisting of:
1-cyclohexylethan-1-yl butyrate;
1-cyclohexylethan-1-yl acetate;
1-cyclohexylethan-1-ol;
1-(4′-methylethyl)cyclohexylethan-1-yl propionate; and
2′-hydroxy-1′-ethyl(2-phenoxy)acetate.

9. The process of claim 1 wherein the scaled aesthetically-pleasing fragrance intensity, Y of the functional substance vs. time in days. X, subsequent to rubbing is defined according to the algorithm:
Y=αX³+βX²+γX+δ
wherein
−0.05≦α≦+0.03;
−2.0≦β≦+0.3;
−1.0≦γ≦+5.0; and
−1.0≦δ≦+5.0.

10. The process of claim 1 wherein at least a finite portion of the microcapsules is in contact with a polymeric silicone phospholipid.

11. The process of claim 10 wherein the polymeric silicone phospholipid is prepared by the phosphation reaction of a terminal dimethicone copolyol with a phosphating agent followed by neutralization of the phosphate with base followed by a condensation reaction with an epihalohydrin followed by conducting a n-alkylation reaction with an amine.

12. A microcapsule consisting essentially of a melamine-formaldehyde polymeric shell and enclosed within the shell a composition comprising a mixture of zinc ricinoleate or a solution thereof and a substituted monocyclic organic compound selected from the group consisting of:
1-cyclohexylethan-1-yl butyrate;
1-cyclohexylethan-1-yl acetate;
1-cyclohexylethan-1-ol:
1-(4′-methylethyl)cyclohexylethan-1-yl propionate; and
2′-hydroxy-1′-ethyl(2-phenoxy)acetate.

13. A slurry comprising an aqueous base, a microcapsule suspended in said base consisting essentially of a melamine-formaldehyde polymeric shell; enclosed within the shell a functional ingredient selected from the group consisting of (a) a fragrance composition each of the components of which has a C log₁₀P of between 2.5 and 8, wherein P is the n-octanol/water partition coeffiicent of said component, and (b) a malodour counteracting composition each of the components of which has a C log₁₀P of between 2.5 and 8, wherein P is the n-octanol/water partition coeffiicent of said component, and in contact with said microcapsule, a polymeric silicone phospholipid.

14. The slurry of claim 13 wherein the polymeric silicone phospholipid is prepared by the phosphation reaction of a terminal dimethicone copolyol with a phosphating agent followed by neutralization of the phosphate with base followed by a condensation reaction with an epihalohydrin followed by conducting a n-alkylation reaction with an amine.

15. The process of claim 1 wherein the aqueous solution provided in step iv consists essentially of (a) from about 80 to about 93 parts by weight of water; (b) from about 4 to about 8 parts by weight of ethanol; (c) from about 2 to about 3 parts by weight of non-ionic surfactant; (d) from about 0.05 to about 0.5 parts by weight of preservative; (e) from about 0.1 to about 2 parts by weight of silicone polymer and (f) from about 0.05 to about 0.1 parts by weight of suspending agent.

16. The process of claim 15 wherein the non-ionic surfactant is a mixture of the hydroxy-octaethoxy ethers of n-nonanol and n-undecanol; the preservative is hexahydro-1,3,5-tris(2-hydroxyethyl)-s-triazine and the suspending agent is selected from the group consisting of attapulgite clay, xanthan gum, hydroxypropyl cellulose having a molecular weight of from about 50,000 to about 800,000, colloidal silica, ethyl cellulose having a particle size of from about 0.004 microns to about 0.130 microns, a surface area of from about 100 m² per gram to about 500 m² per gram and a density of from about 1.0 to about 4.0 pounds per ft³.

17. The slurry of claim 13 wherein the aqueous base consists essentially of (a) from about 80 to about 93 parts by weight of water; (b) from about 4 to about 8 parts by weight of ethanol; (c) from about 2 to about 3 parts by weight of non-ionic surfactant; (d) from about 0.05 to about 0.5 parts by weight of preservative; (e) from about 0.1 to about 2 parts by weight of silicone polymer and (f) from about 0.05 to about 0.1 parts by weight of suspending agent.

18. The slurry of claim 17 wherein the non-ionic surfactant is a mixture of the hydroxy-octaethoxy ethers of n-nonanol and n-undecanol; the preservative is hexahydro-1,3,5-tris(2-hydroxyethyl)-s-triazine and the suspending agent is selected from the group consisting of attapulgite clay, xanthan gum, hydroxypropyl cellulose having a molecular weight of from about 50.000 to about 800,000, colloidal silica, ethyl cellulose having a particle size of from about 0.004 microns to about 0.130 microns, a surface area of from about 100 m² per gram to about 500 m² per gram and a density of from about 1.0 to about 4.0 pounds per ft³.

19. A slurry comprising an aqueous base, admixed with said base a non-confined fragrance composition each of the components of which has a C log₁₀P of between 1 and 8, wherein P is the n-octanol/water partition coeffiicent of said component, a microcapsule suspended in said base consisting essentially of a melamine-formaldehyde polymeric shell; enclosed within the shell a functional ingredient selected from the group consisting of (a) a fragrance composition each of the components of which has a C log₁₀P of between 2.5 and 8, wherein P is the n-octanol/water partition coeffiicent of said component, and (b) a malodour counteracting composition each of the components of which has a C log₁₀P of between 2.5 and 8, wherein P is the n-octanol/water partition coeffiicent of said component, and in contact with said microcapsule and said non-confined fragrance composition, a polymeric silicone phospholipid.

20. The slurry of claim 19 wherein the aqueous base consists essentially of (a) from about 80 to about 93 parts by weight of water; (b) from about 4 to about 8 parts by weight of ethanol; (c) from about 2 to about 3 parts by weight of non-ionic surfactant; (d) from about 0.05 to about 0.5 parts by weight of preservative; (e) from about 0.1 to about 2 parts-by weight of silicone polymer and (f) from about 0.05 to about 0.1 parts by weight of suspending agent.

21. The slurry of claim 20 wherein the non-ionic surfactant is a mixture of the hydroxy-octaethoxy ethers of n-nonanol and n-undecanol; the preservative is hexahydro-1,3,5-tris(2-hydroxyethyl)-s-triazine and the suspending agent is selected from the group consisting of attapulgite clay, xanthan gum, hydroxypropyl cellulose having a molecular weight of from about 50,000 to about 800,000, colloidal silica, ethyl cellulose having a particle size of from about 0.004 microns to about 0.130 microns, a surface area of from about 100 m² per gram to about 500 m² per gram and a density of from about 1.0 to about 4.0 pounds per ft³.
